(19) United States
(12) Patent Application Publication
(10) Pub. No.: US 2023/0250450 A1
(43) Pub. Date: Aug. 10, 2023

(54) NRF2 Activator for Use in Treating Dilated Cardiomyopathies

(71) Applicants: Genethon, Evry-Courcouronnes (FR); Universite d'Evry Val d'Essonne, Evry (FR); INSTITUT NATIONAL DE LA SANTÉ ET DE LA RECHERCHE MÉDICALE, Paris (FR)

(72) Inventor: Isabelle Richard, Corbeil-Essonnes (FR)

(73) Assignees: Genethon, Evry-Courcouronnes (FR); Universite d'Evry Val d'Essonne, Evry (FR); Institut National de la Santé et de la Recherche Médicale, Paris (FR)

(21) Appl. No.: 18/009,025

(22) PCT Filed: Jun. 9, 2021
(86) PCT No.: PCT/EP2021/065430

§ 371 Date: Dec. 8, 2022

(30) Foreign Application Priority Data
Jun. 9, 2020 (EP) 20305620.5

(51) Int. Cl. C12N 15/86; A61P 9/10; A61K 38/17

(52) U.S. Cl. CPC C12N 15/86; A61P 9/10; A61K 38/1709; C12N 2750/14143; C12N 2750/14171; A61K 48/00

(57) ABSTRACT

The present invention relates to the treatment of dilated cardiomyopathies, in particular to the use of an activator of NRF2.

### FIELD OF THE INVENTION

The present invention relates to the treatment of dilated cardiomyopathies, in particular to the use of an activator of NRF2.

### BACKGROUND OF THE INVENTION

Cardiomyopathy and heart failure remain, despite management, one of the major causes of morbidity and mortality worldwide. Dilated cardiomyopathies (DCM or CMD) are characterized by hypokinesis of the myocardium and dilatation of the cardiac cavities. The cardiac remodelling that takes place during dilated cardiomyopathies consists of damage to the cardiomyocytes associated with the presence of fibrosis, which are inseparable from each other. The damage to the cardiomyocytes involves a decrease in their contractile capacity and a change in their structure, which leads to apoptosis and to the expansion of fibrosis, which replaces the necrotic cardiomyocytes. The proliferation of fibroblasts prevents compensatory hypertrophy of the cardiomyocytes. These manifestations will clinically translate into a decrease in cardiac function. This serious complication can be a cause of death.

Causes include in particular genetics, and a variety of toxic, metabolic or infectious agents. Coronary artery disease and high blood pressure may play a role, but are not the primary cause. In many cases the cause remains unclear. The exact mechanism of cardiomyocyte involvement depends on the etiology of the disease. In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RYR2, ATP2A2, phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength. In Duchenne muscular dystrophy (DMD), a muscle disease due to mutation in the dystrophin gene, a dilated cardiomyopathy is clinically revealed around the age of 15 years and affects almost all patients after the age of 20 years. In the case of Becker muscular dystrophy (BMD), an allelic form of DMD, cardiac damage develops at the age of 20 years, and 70% of patients are affected after the age of 35. DMC due to titin, a giant protein of the sarcomere, is implicated in 1/250 cases of heart failure (Burke et al., JCI Insight. 2016;1(6): e86898).).

The drugs currently available for the treatment of dilated cardiomyopathies will improve the symptoms but not treat the causative mechanisms of the disease. The treatments prescribed are those for heart failure, accompanied by hygienic and dietetic measures such as reducing alcohol consumption, reducing water and salt intake and moderate and regular physical exercise. Among pharmaceutical treatments, angiotensin II converting enzyme inhibitors (ACE inhibitors) prevent the production of angiotensin II in order to decrease vasoconstriction and blood pressure. Diuretic drugs remove excess salt and water from the body by inhibiting renal sodium reabsorption. β-blockers or β-adrenergic receptor antagonists, block the effects of adrenergic system mediators stimulated during dilated cardiomyopathies and decrease heart rate. Mineral-corticoid receptor antagonists block the binding of aldosterone and lower blood pressure. When rhythm disturbances are severe, anti-arrhythmic drugs such as amiodarone are prescribed. Implantation of a pacemaker and/or automatic defibrillator may also be considered. In the most severe cases, patients may benefit from a heart transplant (Ponikowski, et al. 2016, European Heart Journal, 37, 2129-2200).

These approaches are therefore also valid for the management of dilated cardiomyopathies in cases of DMD and titinopathies. There is currently no curative treatment for these pathologies. Corticosteroid treatment, frequently prescribed in DMD, allows an improvement of the muscular phenotype in the medium term thanks to a reduction in inflammation, but its action on the cardiac phenotype is subject to debate. The management of DMD related to dystrophin and titin requires an annual and systematic cardiac check-up (electrocardiogram and ultrasound). In particular, Perindopril, an angiotensin-converting enzyme inhibitor, has been shown to reduce mortality in DMD patients when taken as a preventive treatment from childhood onwards (Duboc, D., et al. 2005. Journal of the American College of Cardiology 45, 855-857).

Molecules being tested in the treatment of cardiac impairment in DMD are mainly molecules already used in the treatment of heart failure. Other therapies are aimed at treating muscle and heart damage by reducing fibrosis. This is the case for Pamrevlumab (Phase II trial NCT02606136), a monoclonal antibody directed against connective tissue growth factor, and Tamoxifen (Phase I trial NCT02835079 and Phase III trial NCT03354039), an anti-estrogen. There is therefore a medical need to develop new therapeutic strategies for dilated cardiomyopathies.

The Nuclear factor erythroid-related factor 2 (NRF2) also known as nuclear factor erythroid-derived-2-like 2 is transcription factor that is encoded by the NFE2L2 gene. NRF2 is a basic leucine zipper protein that regulates the expression of antioxidant proteins that protect against oxidative damage triggered by injury and inflammation (Baird, L. and Dinkova-Kostova, A. T, 2011, Arch Toxicol 85, 241-272). Under normal conditions, NRF2 is kept in the cytoplasm by Keap1 and cullin 3 (Cul3), which degrades NRF2 by ubiquitination in the proteasome. Oxidative stress or electrophilic stress disrupts critical cysteine residues in Keap1, which leads to the disruption of the Keap1-Cul3 ubiquitination system. When NRF2 is not ubiquitinated, it is translocated into the nucleus where it could combine with one of the small Maf proteins (MAFF, MAFG, MAFK) and binds to AREs in the upstream promoter region of many anti-oxidative genes to stimulate transcription of anti-oxidative genes (Zhi-Dong Ge et al. 2019, Int Heart J; 60(3):512-520).

NRF2 activity is regulated by many mechanisms, suggesting that tight control is necessary for normal cell function and both hypoactivation and hyperactivation of NRF2 are indicated in playing a role in different aspects of cardiovascular disease. In particular, several studies have shown that NRF2 activation could reduce fibrosis, particularly by inhibiting the ROS/TGFβ1/Smad2/3 pathway (Cai, S. A., et al. 2018, Frontiers in pharmacology 9; Chen, R.-R., et al. 2019, Chemico-Biological Interactions 302, 11-21; Xian, S. et al. 2020. Exp Ther Med 19, 2067-2074). NRF2 degradation has been suggested to be related to a dilated cardiomyopathy phenotype in mice (Jiang, X. et al. 2018. Redox Biol, 19, 134-146). However, the use of NRF2 activator to treat specifically dilated cardiomyopathy has never been disclosed.

### SUMMARY OF THE INVENTION

The inventors developed and characterized a mouse model of genetically-induced dilated cardiomyopathy DeltaMex5 in which the penultimate exon of titin is deleted. Using the DeltaMex5 mice model which is a severe model of dilated cardiomyopathies, the inventors have shown that the overexpression of NRF2 induces significant improvement in cardiac fibrosis and heart hypertrophy indicated that activation of NRF2 represents a therapeutic approach for dilated cardiomyopathy, in particular genetically-induced cardiomyopathy such as titinopathy for which gene transfer approaches are not possible because of the size of the gene.

The present invention relates to a NRF2 activator for use in the treatment dilated cardiomyopathies, preferably a nucleic acid construct comprising a transgene encoding human NRF2 or a variant. In a preferred embodiment, said nucleic acid construct comprises a cardiac promoter selected from the group consisting of: human cardiac troponin T promoter (TNNT2), alpha myosin heavy chain promoter (α-MHC), myosin light chain 2v promoter (MLC-2v), myosin light chain 2a promoter (MLC-2a), CARP gene promoter, alpha-cardiac actin promoter, alpha-tropomyosin promoter, cardiac troponin C promoter, cardiac myosin-binding protein C promoter, sarco/endoplasmic reticulum Ca²⁺ ATPase (SERCA) promoter, desmin promoter, MH promoter, CK8 promoter and MHCK7 promoter, preferably human cardiac troponin T promoter. In a particular embodiment, said nucleic acid construct is packaged into a viral particle, preferably an adeno-associated viral (AAV) particle. Said nucleic acid construct packaged into AAV particle preferably comprises 5′-ITR and 3′-ITR of AAV-2 serotype or a 5′ITR and a 3′ITR corresponding to the serotype of the selected AAV particle. In a particular embodiment, said AAV particle preferably comprises an AAV capsid protein derived from AAV serotypes selected from the group consisting of: AAV-1, 6, 8, 9 and AAV9.rh74 serotypes, more preferably AAV-9.rh74 serotype. In a particular embodiment, said viral particle is administrated intravenously.

According to the present invention, said dilated cardiomyopathy is a genetically induced cardiomyopathy caused by mutation(s) in a gene selected from the group consisting of: laminin, emrin, fukutin, fukutin-related protein, desmocollin, plakoglobin, ryanodine receptor 2, sarcoplasmic reticulum ca(2+) ATPase 2 isoform alpha, phospholamban, lamin a/c, dystrophin, telethonin, actinin, desmin, sarcoglycans, titin, myosin, RNA-binding motif protein 20, BCL-2 associated athanogene 3, desmoplakin, sodium channel, cardiac actin, cardiac troponin and tafazzin, preferably caused by mutation in titin or dystrophin gene.

In another aspect, the present invention relates to a pharmaceutical composition comprising the NRF2 activator as described above and a pharmaceutical excipient.

### DETAILED DESCRIPTION OF THE INVENTION

The present disclosure relates to a NRF2 activator for use in treating a dilated cardiomyopathy in a subject in need thereof.

By “NRF2 activator” is meant any agent that increases NRF2 expression and/or biological activity, in particular that result in a stimulated and/or increased nuclear translocation of NRF2 protein and cause the subsequent increases in target genes such as NAD(P)H quinone oxidoreductase 1 (Nqo1), Glutamate-cystein ligase catalytic subunit (GCLC), Sulfiredoxin 1 (SRXN1), Thioredoxin reductase 1 (TXNRD1), Heme oxygenase-1 (HMOX-1), Glutathione S-transferase (GST), UDP-glucoronosyltransferase (UGT). In particular, said NRF2 activator corresponds to KEAP1 inhibitors.

The NRF2 expression and/or activity can be increased by agents including, but are not limited to, chemicals, compounds known to modify gene expression, modified or unmodified polynucleotides (including oligonucleotides), polypeptides, peptides, small RNA molecules and miRNAs. Such agents are well-known in the art.

The increase of the NRF2 activity may be determined by detecting the NRF2 nuclear translocation in a cell treated with said agent by any well methods in the art such as immunohistochemistry or western blot. The increase of the NRF2 activity may also be determined by measuring the expression level of target genes of NRF2 in a cell treated with said NRF2 activator. In a more particular embodiment, the target gene is selected from the group consisting of NAD(P)H quinone oxidoreductase 1 (Nqo1), Glutamate-cystein ligase catalytic subunit (GCLC), Sulfiredoxin 1 (SRXN1), Thioredoxin reductase 1 (TXNRD1), Heme oxygenase-1 (HMOX-1), Glutathione S-transferase (GST), UDP-glucoronosyltransferase (UGT). The NRF2 activity is increased in cells when the expression level of the target gene is at least 1.5-fold lower, or 2, 3, 4, 5-fold lower than in non-treated cells.

The expression level of mRNA may be determined by any suitable methods known by skilled persons. For example the nucleic acid contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR).

The level of the target genes protein may also be determined by any suitable methods known by skilled persons. The quantity of the protein may be measured, for example, by semi-quantitative Western blots, enzyme-labelled and mediated immunoassays, such as ELISAs, biotin/avidin type assays, radioimmunoassay, immunoelectrophoresis, mass spectrometry, or immunoprecipitation or by protein or antibody arrays.

In another particular embodiment, the increase of NRF2 activity may be determined by measuring the expression level of NRF2. The regulatory activity of the NRF2 is increased in cells when the expression level of NRF2 is at least 1.5-fold higher, or 2, 3, 4, 5-fold higher than in non-treated cells. The expression level of NRF2 can be determined by any suitable methods known by skilled persons as described above.

NRF2 activators are well-known in the art and can be selected from the group consisting of electrophilic compounds, protein-protein interaction (PPI) inhibitors, and multi-target drugs. In a particular embodiment, said NRF2 activators are electrophilic compounds which can be selected as non-limiting examples from the group consisting of: bardoloxone-methyl (CDDO-Me), RTA-408 (omaveloxolone), Dimethyfumarate, ALKS-8700, Oltipraz, Ursodiol, Sulforaphane, Sulforadex (SFX-01), ITH12674, curcumin, resveratrol, quercetin, genistein, andeographolide and CXA-10. NRF2 activators can also be protein-protein interaction inhibitors selected as non-limiting examples from the group consisting of: peptides, RS-5,benzenesulfonyl-pyrimidone 2,N-phenyl-benzenesulfonamide and 1,4-diphenyl-1,2,3-triazole, or canonical NRF2 activators selected from the group consisting of sulforaphane (SFN) and tert-butyl hydroquinone (tBHQ).

### Transgene

In a preferred embodiment, the present disclosure relates to the treatment of dilated cardiomyopathy by gene therapy. The term “gene therapy” refers to treatment of a subject which involves delivery of a gene/nucleic acid into an individual's cells for the purpose of treating a disease. Delivery of the gene is generally achieved using a delivery vehicle, also known as a vector. Viral and non-viral vectors may be employed to deliver a gene to a patient's cells.

Thus, in a particular embodiment, the NRF2 activator is a transgene encoding NRF2 or a variant thereof in a subject.

As used herein, the term “transgene” refers to exogenous DNA or cDNA encoding a gene product. The gene product may be an RNA, peptide or protein. In addition to the coding region for the gene product, the transgene may include or be associated with one or more elements to facilitate or enhance expression, such as a promoter, enhancer(s), response element(s), reporter element(s), insulator element(s), polyadenylation signal(s) and/or other functional elements. Embodiments of the disclosure may utilize any known suitable promoter, enhancer(s), response element(s), reporter element(s), insulator element(s), polyadenylation signal(s) and/or other functional elements. Suitable elements and sequences will be well known to those skilled in the art.

The terms “nucleic acid sequence” and “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholines and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-0-allyl analogs and 2′-0-methylribonucleotide methylphosphonates which may be used in a nucleotide of the disclosure.

The transgene according to the disclosure may be any nucleic acid sequence encoding an NFR2 protein, in particular a native mammalian, preferably human, NFR2 protein, or a variant thereof.

The gene Nuclear factor, erythroid 2 like 2 (NFE2L2) (Gene ID: 4780), also known as NRF2, HEBP1 encodes for six human NRF2 protein isoforms, isoform 1 (accession number: NP_006155.2), isoform 2 (accession number: NP_001138885.1), isoform 3 (accession number: NP_001138885.1), isoform 4 (accession number: NP_001300831.1), isoform 5 (accession number: NP_001300832.1) and isoform 6 (accession number: NP_001300833.1). Preferably, Human NRF2 protein comprises, or consists of, the amino acid sequence of isoform 1 or A.

The coding sequences of a number of different mammalian NRF2 proteins are known including, but being not limited to, human, pig, chimpanzee, dog, cow, mouse, rabbit or rat, and can be easily found in sequence databases. Alternatively, the coding sequence may be easily determined by the skilled person based on the polypeptide sequence.

In a preferred embodiment, said transgene comprises coding sequence for NRF2 protein which can be selected from the group consisting of the reference sequences of the human nuclear factor, erythroid 2 like 2 (NFE2L2) transcript variant 1 (accession number: NM_006164.5), transcript variant 2 (accession number: NM_001145412.3), transcript variant 3 (accession number: NM_001145413.3), transcript variant 4 (accession number: NM_001313900.1), transcript variant 5 (accession number: NM_001313901.1), transcript variant 6 (accession number: NM_001313902.1), transcript variant 7 (accession number: NM_001313903.1) and transcript variant 8 (accession number: NM_001313904.1).

In a particular embodiment, the transgene according to the disclosure may be any nucleic acid sequence encoding an NFR2 protein variant.

Preferably, as used herein, the term “variant” refers to a polypeptide having an amino acid sequence having at least 70, 75, 80, 85, 90, 95 or 99% sequence identity to the native sequence. As used herein, the term “sequence identity” or “identity” refers to the number (%) of matches (identical amino acid residues) in positions from an alignment of two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al, 1997; Altschul et al., 2005). Alignment for purposes of determining percent amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

More preferably, the term “variant” refers to a polypeptide having an amino acid sequence that differs from a native sequence by less than 30, 25, 20, 15, 10 or 5 substitutions, insertions and/or deletions. In a preferred embodiment, the variant differs from the native sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine). NRF2 activity of a variant may be assessed by any method known by the skilled person as described above.

In a particular embodiment said transgene may be an optimized sequence encoding NRF2 protein or variant thereof.

The term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein.

### Nucleic Acid Construct

In a particular embodiment, said transgene is comprised in a nucleic acid construct.

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

Preferably, the nucleic acid construct comprises the transgene operably linked to one or more control sequences that direct the expression of said transgene in cardiac cells.

The control sequence may include a promoter that is recognized by cardiac cells. The promoter contains transcriptional control sequences that mediate the expression of NRF2 protein upon introduction into a host cell. The promoter may be any polynucleotide that shows transcriptional activity in cells including mutant, truncated, and hybrid promoters. The promoter may be a constitutive or inducible promoter, preferably a constitutive promoter, and more preferably a strong constitutive promoter.

The promoter may also be tissue-specific, in particular specific of cardiac cells. In a particular embodiment, the nucleic acid construct of the disclosure further comprises a cardiac-specific promoter operably-linked to the transgene as described above. In the context of this disclosure, a “cardiac-specific promoter” is a promoter which is more active in the cardiac than in any other tissue of the body. Typically, the activity of a cardiac specific promoter will be considerably greater in the cardiac than in other tissues. For example, such a promoter may be at least 2, at least 3, at least 4, at least 5 or at least 10 times more active (for example as determined by its ability to drive the expression in a given tissue in comparison to its ability to drive the expression in other cells or tissues). Accordingly, a cardiac specific promoter allows an active expression in the cardiac of the gene linked to it and prevents its expression in other cells or tissues.

Examples of suitable promoters include, but are not limited to, human troponin T gene promoter (TNNT2), alpha myosin heavy chain promoter (α-MHC), myosin light chain 2 promoter (MLC-2), alpha-cardiac actin promoter, alpha-tropomyosin promoter, cardiac troponin C promoter, cardiac myosin-binding protein C promoter, sarco/endoplasmic reticulum Ca²⁺ ATPase (SERCA) promoter, desmin promoter, MH promoter, CK8 promoter and MHCK7 promoter. Preferably, the promoter is the human cardiac troponin T promoter. The Muscle Hybrid promoter (MH promoter) is disclosed for example in Piekarowicz et al., Molecular Therapy, 2019, 15, 157-169). CK8 is muscle creatine kinase promoter/enhancer element (Gonçalves et al., Mol. Ther., 2011, 19, 1331-1341). MHCK7 promoter is based on enhancer/promoter regions of muscle creatine kinase (CK) and alpha-myosin heavy-chain genes (Salva et al., Mol. Ther., 2007, 15, 320-329).

The control sequence may also include appropriate transcription initiation, termination, and enhancer sequences; efficient RNA processing signals such as splicing and polyadenylation signals; sequences that stabilize cytoplasmic mRNA; sequences that enhance translation efficiency (i.e., Kozak consensus sequence); and/or sequences that enhance protein stability. A great number of expression control sequences, e.g., native, constitutive, inducible and/or tissue- specific, are known in the art and may be utilized to drive expression of the nucleic acid sequence encoding NRF2. Typically, the transgene encoding NRF2 is operably linked to a transcriptional promoter and a transcription terminator.

Apart from the specific delivery systems embodied below in the examples, various delivery systems are known and can be used to administer the nucleic acid construct as described above, e.g., encapsulation in liposomes, microparticles, microcapsules, recombinant cells capable of expressing the NRF-2 coding sequence, receptor-mediated endocytosis, construction of a therapeutic nucleic acid as part of a retroviral or other vector, etc.

### Expression Vector

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected.

The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

Thus, in one embodiment, the nucleic acid construct comprising transgene as described above further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the nucleic acid construct of the disclosure may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype or engineered AAV now known or later discovered.

In one embodiment, the nucleic acid construct further comprises a 5′ITR and a 3′ITR of an AAV-2 serotype or a 5′ITR and a 3′ITR corresponding to the serotype of the selected AAV particle

On the other hand, the nucleic acid construct as described above can be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the disclosure comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

### Viral Particle

In a particular embodiment, the present disclosure relates to viral particles including the nucleic acid construct or expression vector as described above.

The nucleic acid construct or the expression vector of the disaclosure may be packaged into a virus capsid to generate a “viral particle”, also named “viral vector particle”. In a particular embodiment, the nucleic acid construct or the expression vector as described above is packaged into an AAV-derived capsid to generate an “adeno-associated viral particle” or “AAV particle”. The present disclosure relates to a viral particle comprising a nucleic acid construct or an expression vector of the disclosure and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype on a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bünning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in AAV viral particle according to the present disclosure, the nucleic acid construct or viral expression vector including ITR(s) of a given AAV serotype can be packaged, for example, into: a) a viral particle constituted of capsid proteins derived from the same or different AAV serotype; b) a mosaic viral particle constituted of a mixture of capsid proteins from different AAV serotypes or mutants; c) a chimeric viral particle constituted of capsid proteins that have been truncated by domain swapping between different AAV serotypes or variants.

The skilled person will appreciate that the AAV viral particle for use according to the present disclosure may comprise capsid proteins derived from any AAV serotype including AAV1, AAV2, AAV3 (including types 3A and 3B), AAV4, AAV5, AAV6, AAV7, AAV8, AAV9, AAV10, AAV11, AAV12, AAV2i8, AAVrh10, AAVrh39, AAVrh43, AAVrh74, AAV-LK03, AAV2G9, AAV.PHP, AAV-Anc80, AAV3B and AAV9.rh74 (as disclosed in WO2019/193119).

For gene transfer into human cardiac cells, AAV serotypes 1, 6, 8, 9 and AAV9.rh74 are preferred. The AAV serotype 9 and AAV9.rh74 are particularly well suited for the induction of expression in cells of the myocardium/cardiomyocytes. In a specific embodiment, the AAV viral particle comprises a nucleic acid construct or expression vector of the disclosure and preferably capsid proteins from AAV9 or AAV9.rh74 serotype.

### Pharmaceutical Composition

The NRF2 activator, nucleic acid construct, expression vector or viral particle according to the present disclosure is preferably used in the form of a pharmaceutical composition comprising a therapeutically effective amount of NRF2 activator, nucleic acid construct, expression vector or viral particle according to the present disclosure.

In the context of the disclosure, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present disclosure, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

### Treatment of Dilated Cardiomyopathies

The NRF2 activator, nucleic acid construct, expression vector or viral particle according to the present disclosure is used for the treatment of any dilated cardiomyopathy (DCM).

Dilated cardiomyopathy (CMD) is characterized by cardiac dilatation and reduced systolic function. CMD is the most frequent form of cardiomyopathy and accounts for more than half of all cardiac transplantations performed in patients between 1 and 10 years of age. Causes of DCMs include in particular genetics, and a variety of toxic, metabolic or infectious agents. Toxic or metabolic agents include in particular alcohol and cocaine abuse and chemotherapeutic agents such as for example doxorubicin and cobalt; Thyroid disease; inflammatory diseases such as sarcoidosis and connective tissue diseases; Tachycardia-induced cardiomyopathy; autoimmune mechanisms; complications of pregnancy; and thiamine deficiency. Infectious agents include in particular Chagas disease due to Trypanosoma cruzi and sequelae of acute viral myocarditis such as for example with Coxsackie B virus and other enteroviruses. A heritable pattern is present in 20 to 30% of cases. Most familial CMD pedigrees show an autosomal dominant pattern of inheritance, usually presenting in the second or third decade of life (summary by Levitas et al., Europ. J. Hum. Genet., 2010, 18: 1160-1165).

In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RyR2, SERCA2a, phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength.

Mutations in many genes have been found to cause different forms of dilated cardiomyopathy (CMD). These include in particular:
CMD1A, dilated cardiomyopathy-1A (OMIM #115200) caused by heterozygous mutation in the lamin A/C gene (LMNA), (OMIM #150330) on chromosome 1q22; or heterozygous mutation in the laminin alpha 2 (LAMA2 or MEROSIN) gene (OMIM #156225; Marques et al., Neuromuscul. Disord., 2014, doi.org/10.10106/);
CMD1B (OMIM #600884) on 9q13; the gene referred as FDC locus was placed in the interval between D9S153 and D9S152. Friedreich ataxia (OMIM #229300), which is frequently associated with dilated cardiomyopathy, maps to the same region as does also the cAMP-dependent protein kinase (OMIM #176893), which regulates calcium-channel ion conductance in the heart. Tropomodulin (OMIM #190930), which maps to 9q22, was a particularly attractive candidate gene.
CMD1C (OMIM #601493) with or without left ventricular noncompaction, caused by mutation in the lim domain-binding 3, LDB3 (or ZASP) gene (OMIM #605906) on 10q23;
CMD1D (OMIM #601494), caused by mutation in the troponin T2, cardiac (TNNT2) gene (OMIM #191045) on 1q32;
CMD1E (OMIM #601154), caused by mutation in the SCN5A gene (OMIM #600163) on 3p22;
CMD1F: The symbol CMD1F was formerly used for a disorder later found to be the same as desmin-related myopathy or myopathy, myofibrillar (MFM) (OMIM #601419);
CMD1G (OMIM #604145), caused by mutation in the titin (TTN) gene (OMIM #188840) on 2q31;
CMD1H (OMIM #604288) on 2q14-q22;
CMD1I (OMIM #604765), caused by mutation in the desmin (DES) gene (OMIM #125660) on 2q35;
CMD1J (OMIM #605362), caused by mutation in the EYA4 gene (OMIM #603550) on 6q23;
CMD1K (OMIM #605582) on 6q12-q16;
CMD1L (OMIM #606685), caused by mutation in the sarcoglycan delta (SGCD) gene (OMIM #601411) on 5q33;
CMD1M (OMIM #607482), caused by mutation in the CSRP3 gene (OMIM #600824) on 11p15;
CMD1N; (OMIM #607487) caused by mutation in the TITIN-CAP (telethonin or TCAP) gene (OMIM #604488).
CMD1O (OMIM #608569), caused by mutation in the ABCC9 gene (OMIM #601439) on 12p12;
CMD1P (OMIM #609909), caused by mutation in the phospholamban (PLN) gene (OMIM #172405) on 6q22;
CMD1Q (OMIM #609915) on 7q22.3-q31.1;
CMD1R (OMIM #613424), caused by mutation in the actin alpha, cardiac muscle (ACTC1) gene (OMIM #102540) on 15q14;
CMD1S (OMIM #613426), caused by mutation in the myosin heavy chain 7, cardiac muscle, beta (MYH7) gene (OMIM #160760) on 14q12;
CMD1U (OMIM #613694), caused by mutation in the PSEN1 gene (OMIM #104311) on 14q24;
CMD1V (OMIM #613697), caused by mutation in the PSEN2 gene (OMIM #600759) on 1q42;
CMD1W (OMIM #611407), caused by mutation in the gene encoding metavinculin (VCL; OMIM #193065) on 10q22;
CMD1X (OMIM #611615), caused by mutation in the gene encoding fukutin (FKTN; OMIM #607440) on 9q31;
CMD1Y (OMIM #611878), caused by mutation in the TPM1 gene (OMIM #191010) on 15q22;
CMD1Z (OMIM #611879), caused by mutation in the troponin C (TNNC1) gene (OMIM #191040) on 3p21;
CMD1AA (OMIM #612158), caused by mutation in the actinin alpha-2 (ACTN2) gene (OMIM #102573) on 1q43;
CMD1BB (OMIM #612877), caused by mutation in the DSG2 gene (OMIM #125671) on 18q12;
CMD1CC (OMIM #613122), caused by mutation in the NEXN gene (OMIM #613121) on 1p31;
CMD1DD (OMIM #613172), caused by mutation in the RNA binding motif protein 20 (RBM20) gene (OMIM #613171) on 10q25;
CMD1EE (OMIM #613252), caused by mutation in the myosin heavy chain 6, cardiac muscle, alpha (MYH6) gene (OMIM #160710) on 14q12;
CMD1FF (OMIM #613286), caused by mutation in the troponin I, cardiac (TNNI3) gene (OMIM #191044) on 19q13;
CMD1GG (OMIM #613642), caused by mutation in the SDHA gene (OMIM #600857) on 5p15;
CMD1HH (OMIM #613881), caused by mutation in the BCL2-associated athanogene 3 (BAG3) gene (OMIM #603883) on 10q26;
CMD1II (OMIM #615184), caused by mutation in the CRYAB gene (OMIM #123590) on 6q21;
CMD1JJ (OMIM #615235), caused by mutation in the laminin alpha 4 (LAMA4) gene (OMIM #600133) on 6q21;
CMD1KK (OMIM #615248), caused by mutation in the MYPN gene (OMIM #608517) on 10q21;
CMD1LL (OMIM #615373), caused by mutation in the PRDM16 gene (OMIM #605557) on 1p36;
CMD1MM (OMIM #615396), caused by mutation in the MYBPC3 gene (OMIM #600958) on 11p11;
CMD1NN (OMIM #615916), caused by mutation in the RAF1 gene (OMIM #164760) on 3p25;
CMD2A (OMIM #611880), caused by mutation in the troponin I, cardiac (TNNI3) gene on 19q13;
CMD2B (OMIM #614672), caused by mutation in the GATAD1 gene (OMIM #614518) on 7q21;
CMD2C (OMIM #618189), caused by mutation in the PPCS gene (OMIM #609853) on 1p34;
CMD3A, a previously designated X-linked form was found to be the same as Barth syndrome (OMIM #302060); and
CMD3B (OMIM #302045), an X-linked form of CMD, caused by mutation in the dystrophin gene (DMD, OMIM #300377).

Desmin-related myopathy or myopathy, myofibrillar (MFM) (OMIM #601419). is a noncommittal term that refers to a group of morphologically homogeneous, but genetically heterogeneous chronic neuromuscular disorders. The morphologic changes in skeletal muscle in MFM result from disintegration of the sarcomeric Z disc and the myofibrils, followed by abnormal ectopic accumulation of multiple proteins involved in the structure of the Z disc, including desmin, alpha-B-crystallin (CRYAB; OMIM #123590), dystrophin (OMIM #300377), and myotilin (TTID; OMIM #604103). Myofibrillar myopathy-1 (MFM1) is caused by heterozygous, homozygous, or compound heterozygous mutation in the desmin gene (DES; OMIM #125660) on chromosome 2q35. Other forms of MFM include MFM2 (OMIM #608810), caused by mutation in the CRYAB gene (OMIM #123590); MFM3 (OMIM #609200) (OMIM #182920), caused by mutation in the MYOT gene (OMIM #604103); MFM4 (OMIM #609452), caused by mutation in the ZASP gene (LDB3; OMIM #605906); MFMS (OMIM #609524), caused by mutation in the FLNC gene (OMIM #102565); MFM6 (OMIM #612954), caused by mutation in the BAG3 gene (OMIM #603883); MFM7 (OMIM #617114), caused by mutation in the KY gene (OMIM #605739); MFM8 (OMIM #617258), caused by mutation in the PYROXD1 gene OMIM #617220); and MFM9 (OMIM #603689), caused by mutation in the TTN gene (titin ; OMIM #188840).

Mutations in other genes have also been found to cause different forms of dilated cardiomyopathy. These include:
desmocollin 2 (DSC2, OMIM #125645) responsible for Arrhythmogenic Right Ventricular Dysplasia 11 (OMIM #610476) and dilated cardiomyopathy (Elliott et al., Circ. Vasc. Genet., 2010, 3, 314-322);
junctiun plakoglobin (JUP or plakoglobin; OMIM #173325) responsible for Arrhythmogenic Right Ventricular Dysplasia 12 (OMIM #611528) and dilated cardiomyopathy (Elliott et al., Circ. Vasc. Genet., 2010, 3, 314-322);
ryanodine receptor 2 (RYR2; OMIM #180902) responsible for Arrhythmogenic Right Ventricular Dysplasia 2 (OMIM #600996) and Ventricular tachycardia, catecholaminergic polymorphic 1 (OMIM #604772) and dilated cardiomyopathy (Zahurul, Circulation, 2007, 116, 1569-1576);
ATPase, Ca(2+)-transporting slow-twitch (ATP2A2; ATP2B, sarcoplasmic reticulum Ca(2+) ATPase 2 isoform alpha (SERCA2a).
emerin (EMD); fukutin-related protein (FKRP); tafazzin (TAZ); desmoplakin (DSP); and Sodium Channels such as SCN1B, SCN2B, SCN3B, SCN4B, SCN4A, SCN5A and others.

In some embodiments, the dilated cardiomyopathy is an acquired dilated cardiomyopathy; for example caused by toxic, metabolic or infectious agents according to the present disclosure. The cause of the dilated cardiomyopathy may also be unknown (idiopathic dilated cardiomyopathy).

In some preferred embodiments, the dilated cardiomyopathy is a genetic dilated cardiomyopathy; preferably caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); lamin A/C (LMNA); dystrophin (DMD); TITIN-CAP or telethonin (TCAP); actinin, in particular actinin alpha-2 (ACTN2); desmin (DES); actin, in particular cardiac actin, actin alpha, cardiac muscle (ACTC1); sarcoglycan, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others, preferably dystrophin (DMD) or titin (TTN)

The disclosure provides also a method for treating a dilated cardiomyopathy according to the present disclosure, comprising: administering to a patient a therapeutically effective amount of the NRF2 activator, nucleic acid construct or viral particle or pharmaceutical composition as described above.

The disclosure provides also the use of the NRF2 activator, nucleic acid construct or viral particle or pharmaceutical composition as described above for the treatment of a dilated cardiomyopathy according to the present disclosure.

The disclosure provides also the use of the NRF2 activator, nucleic acid construct or viral particle or pharmaceutical composition as described above in the manufacture of a medicament for treatment of a dilated cardiomyopathy according to the present disclosure.

The disclosure provides also a pharmaceutical composition for treatment of a dilated cardiomyopathy according to the present disclosure, comprising the NRF2 activator, nucleic acid construct or viral particle as described above as an active component.

The disclosure provides also a pharmaceutical composition comprising the NRF2 activator, nucleic acid construct or viral particle as described for treating a dilated cardiomyopathy according to the present disclosure,

By “therapeutically effective amount” refers to an amount effective, at dosages and for periods of time necessary to achieve the desired therapeutic result, such as reduction of cardiac fibrosis and/or improve of cardiac function. The therapeutically effective amount of the product of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

As used herein, the term “patient” or “individual” denotes a mammal. Preferably, a patient or individual according to the disclosure is a human.

In the context of the disclosure, the term “treating” or “treatment”, as used herein, means reversing, alleviating or inhibiting the progress of the dilated cardiomyopathy or condition to which such term applies, or reversing, alleviating or inhibiting the progress of one or more symptoms of the disorder or condition to which such term applies, in particular reduction of cardiac fibrosis and/or improve of cardiac function.

The pharmaceutical composition of the present disclosure is generally administered according to known procedures, at dosages and for periods of time effective to induce a therapeutic effect in the patient.

The administration can be systemic or local. Systemic administration is preferably parenteral such as subcutaneous (SC), intramuscular (IM), intravascular such as intravenous (IV) or intraarterial; intraperitoneal (IP); intradermal (ID), interstitial or else. The administration may be for example by injection or perfusion. In some preferred embodiments, the administration is parenteral, preferably intravascular such as intravenous (IV) or intraarterial. The practice of the present disclosure will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Morphological analysis. A) Total mass of mice. B) Measurement of cardiac hypertrophy: heart mass/total mouse mass (%).

FIG. 2: Histological characterization of the heart after injection of AAV9-tnnt2-NRF2 in DeltaMex5 mice and controls injected with PBS. A) HPS staining of the heart. B) Sirius red staining of the heart, Scale, 500 μm. C) Sirius quantification. Student's test.

### EXAMPLES

### 1.1 Mouse Models

The mice used in this study were male titinMex5-/Mex5- (DeltaMex5) and DBA/2J-mdx (DBA2mdx) strains, and their respective controls, strains C57BL/6 and DBA/2. DeltaMex5 mice are mice in which the penultimate exon of Titin is deleted by CRISPR-Cas9 technology (Charton, K., et al. 2016, Human molecular genetics 25, 4518-4532). DBA2mdx mice are a model of Duchenne muscular dystrophy due to a point mutation on exon 23 of the dystrophin gene. DBA2mdx mice are on a DBA/J background which has a mutation on the LTBP4 gene, a protein that regulates the activity of the TGF signalling pathway-β (Fukada, et al. 2010. Am J Pathol 176, 2414-2424). All the mice are handled in accordance with the

European directives for the care and use of laboratory animals by humans, and the animal experimentation has been approved by the Ethics Committee for Animal Experimentation C2AE-51 of Evry under the numbers of Project Authorisation Application 2015-003-A and 2018-024-B.

### 1.2 Muscle Sampling and Freezing

The muscles of interest are collected, weighed and frozen in liquid nitrogen (samples for molecular biology analysis) or in cooled isopentane (samples for histology), after being placed transversely or longitudinally on a piece of cork coated with gum arabic. The hearts are frozen in diastole before being frozen with a diluted butanedione solution (5 mM) in tyrode. The samples are then stored at −80° C. until use. For the Sirius Red Fibrosis observation protocol on the whole heart, the hearts are included whole in paraffin and stored at room temperature. For the transparency protocols, the sampled hearts are stored whole in 4% para formaldehyde and kept at +4° C.

### 1.3 Haematoxylin-Phloxine-Safran Staining

The Hematoxylin-Phloxine-Safran (HPS) marking allows to observe the general appearance of the muscle and to highlight the different tissue and cell structures. Haematoxylin colours nucleic acids dark blue, phloxine colours the cytoplasm pink, saffron colours collagen red-orange. Cross sections are stained with Harris hematoxylin (Sigma) for 5 min. After washing with water for 2 min, the slides are immersed in a 0.2% (v/v) hydrochloric alcohol solution for 10 s to remove excess stain. After being washed again with water for 1 min, the tissues are blued in a Scott water bath (0.5 g/l sodium bicarbonate and 20 g/l magnesium sulfate solution) for 1 min before being rinsed again with water for 1 min and stained with phloxine 1% (w/v) (Sigma) for 30 s. After rinsing with water for 1 min 30 s, the cuts are dehydrated with 70° ethanol for 1 min and then rinsed in absolute ethanol for 30 s. The tissues are then stained with saffron 1% (v/v in absolute ethanol) for 3 min and rinsed in absolute ethanol. Finally, the cuts are thinned in a Xylene bath for 2 min and then mounted with a slide in the Eukitt medium. Image acquisition is performed with objective 10 on a Zeiss AxioScan white light microscope coupled to a computer and a motorized stage.

From HPS coloured sections, the centronucleation index is calculated by the ratio of the number of centronucleated fibres to the area of the section in mm2.

### 1.4 Sirius Red Coloration

Sirius Red staining allows the collagen fibres to be coloured red and to highlight the presence of fibrotic tissue. Cytoplasms are stained yellow. Cross sections are dehydrated with acetone for 1 hour for frozen cuts or dewaxed with heat and toluene baths. They are then fixed with 4% formaldehyde for 5 min then 10 min in a Bouin solution. After two washes with water, the slides are immersed in Sirius Red solution (0.1 g Sirius Red per 100 mL picric acid solution) for 1 h for staining. After rinsing with water for 1 min 30 s, the slices are dehydrated in successive ethanol baths: 70° ethanol for 1 min, 95° ethanol for 1 min, absolute ethanol for 1 min and then a second absolute ethanol bath for 2 min. Finally, the slices are thinned in two Xylene baths for 1 min and then mounted with a lamella in the Eukitt medium. Image acquisition is carried out with objective 10× on a Zeiss AxioScan white light microscope coupled to a computer and a motorized stage. The polarized light images were acquired using a modified right LEICA microscope with a polarizer placed before the sample (Polarizer), along the path of the light; and another polarizer placed after the sample (Anlayzer), which can be rotated by hand, giving the possibility to observe both transmitted and polarized light at the same time. The main axes of the polarizers are oriented at 90 degrees to each other. Polarized light maps have been acquired using a Retiga 2000 CCD sensor (QImaging) coupled to the Cartograph software (Microvision, France). In summary, the light passes through the first polarizer before reaching the sample, the collagen being birefringent the light that passes through it is separated into two rays, which once passed through the second polarizer will allow the differential observation of the two types of Sirius Red and the rest of the cardiac tissue.

### 1.5 Quantification of Sirius Red

### Sirius Quant

Sirius Quant is an internally developed ImageJ pluggin (Schneider et al., 2012). It is a thresholding macro that allows to isolate and quantify the pixels of the image that are colored red. It works in 3 steps: the first one is to convert the image to black and white. The images resulting from the Red Sirius colorations are very contrasted, so a simple black and white conversion is enough to keep all the useful information. The second one is a very rough thresholding in order to keep only the colored pixels of the image, in other words the pixels belonging to the whole cut. Using the Analyze Particles function with an adapted object size allows automatic detection of the outline of the slice, which is then stored. The third step is a manual thresholding by the user which allows to keep only the pixels colored in red, those associated with the marking. A manual correction tool makes it possible either to remove areas that would have been detected and that are not marking (dust, cut fold, etc.), or to add areas that would not have been taken into account. Once the thresholded image is satisfactory, the number of thresholded pixels and the total number of pixels in the entire section are then measured. A ratio between these two numbers finally gives the fibrosis index in the slice.

### WEKA

The images were processed using an artificial intelligence algorithm via the WEKA plugin (ImageJ). The WEKA classifier pluggin was implemented using a training data set containing 17 images representative of the different conditions to be classified. The classes were assigned to healthy tissue (yellow), to both types of staining and to slice rupture (white). The original mappings are mosaic images with a size of approximately 225 megapixels (15k×15k), which were divided into 400 frames (20 rows, 20 columns), each frame measuring approximately 750×750 pixels. Each frame is classified independently and the complete image is then reconstructed. The number of pixels in each class is measured. The total number of pixels belonging to the heart is calculated as the sum of the healthy tissue and the two types of dye uptake. The ratio of each class is then calculated by dividing the number of pixels in the class by the total number of pixels in the heart.

### Whole-Heart Reconstruction and Quantification

Sections of a whole heart colored by Sirius Red were scanned with a scanner (Axioscan ZI, Zeiss) with a 10× lens. A total of 483 images were obtained. They were aligned using Imagers pluggin: Linear Stack Alignment with SIFT (Lowe et al., International Journal of Computer Vision, 2004, 60, 91-110). Some images were manually aligned when the software did not allow a satisfactory alignment. The image was loaded into Imaris (BitPlane, USA) for reconstruction and 3D visualization. Once the images were aligned, the Sirius Quant pluggin in fully automatic mode using Otsu thresholding (Otsu N, Cybernetics, 1979, 9, 62-66) resulted in 483 fibrosis ratio values corresponding to each image. These values were filtered using the sliding average method, which is a method of reducing noise in a signal to avoid the errors inherent in automating an algorithm. The use of the moving average allows to limit these errors by replacing each fibrosis ratio of an image by the average of itself, the ratio of the image preceding it and the ratio of the image following it.

### 1.6 Viral Vectors

The vector AAV9, with a cardiac troponin promoter, carrying the murine transgene encoding Nrf2 (AAV9-tnnt2-mNRF2) was ordered from Vectrobiolabs.

### 1.7 Production of Plasmids

Plasmids are produced by transforming 45 μL of DH10B bacteria with 2 μL of plasmid. Thermal shock is achieved by alternating 5 minutes in ice, 30 seconds at 42° C. and cooling on ice. Then, 250 μL of SOC (super optimal broth) medium is added before incubation at 37° C. for 1 h under agitation. The bacteria thus transformed are isolated by a 50 μL culture over night at 37° C. on a box of LB (lysogeny broth) containing ampicillin in order to select the bacteria having integrated the plasmid. A clone is transplanted the next day for a pre-culture of a few hours at 37° C. in 3 mL of LB medium containing antibiotic. Samples are kept for freezing in 50% glycerol. An overnight culture is then performed in 2L Erlenmeyer containing 500 mL of antibiotic-containing medium and 1 mL of the preculture at 37° C. A NucleoBond PC 2000 EF (Macherey Nagel) kit is then used according to the supplier's instructions to purify the plasmids which are then sterilized by filtration at 0.22 μm and assayed with Nanodrop.

An enzymatic digestion is performed to check the plasmid with the restriction enzymes SMA1 and NHE1. A mixture containing 1 μg of DNA, 2 μL of buffer fast digest green 10×, 1 μl of each enzyme in sterile water for a total amount of 20 μl is stirred for 20 min at 37° C. A 1% agarose gel in TAE (Tris, Acetate, EDTA) containing SYBR™ Safe DNA Gel Stain (Invitrogen) is poured before depositing the digest products and the size marker O'GeneRuler™ DNA Ladder mix.

### 1.8 Vector Production

The tri-transfection method is used to prepare recombinant viruses. HEK293 cells are used as packaging cells to produce the virus particles. Three plasmids are required: the vector plasmid, which provides the gene of interest, the helper plasmid pAAV2-9_Genethon_Kana (Rep2Cap9), which provides the Rep and Cap viral genes, and plasmid pXX6, which contains adenoviral genes and replaces the co-infection by an adenovirus, necessary for AAV replication. The cells are then lysed and the viral particles are purified. Vectors are produced in suspension.

Cell inoculation (day 1): Use of HEK293T clone 17 cells at confluence, inoculated in 1 L agitation flasks: 2E5 cells/mL in 400 mL of F17 medium (Thermo Fisher scientific). Incubation under agitation (100 rpm) at 37° C.—5% CO2—humid atmosphere.

Cell Transfection (Day 3): Cells are counted and cell viability is measured on Vi-CELL after 72 h of culture. The transfection mix is prepared in Hepes buffer at 10 mg/mL for each plasmid according to its concentration, size and the amount of cells in the flask, the ratio of each plasmid is 1. Incubation 30 minutes at RT after the addition of transfection agent and homogenization of the solution. The transfection mixture and 3979 μL of culture medium (F17 GNT Modified) are transferred to shaker flasks containing 400 mL of culture which are incubated under agitation (130 rpm) at 37° C.—5% CO2—wet atmosphere. After 48 h, treatment of the cells with benzonase: dilution of Benzonase (25 U/mL final) and MgCl2 (2 mM final) in F17 medium, addition of 4 mL per flask.

Viral vector harvest (day 6): Cells are counted and cell viability is measured on Vi-CELL, then 2 mL of triton X-100 (Sigma, 1/200th dilution) are added before incubating 2.5 hours at 37° C. with agitation. The erlenmeyers are transferred to Corning 500 mL and centrifuged at 2000 g for 15 minutes at 4° C. Supernatants are transferred to new Corning 500 mL before adding 100 mL of PEG 40%+NaCl and incubating 4 h at 4° C. The suspension is centrifuged at 3500 g for 30 minutes at 4° C. The pellets are resuspended in 20 mL TMS at pH 8 (Tris HCl at 50 mM, NaCl at 150 mM and MgCl2 at 2 mM, diluted in water) and transferred to Eppendorf 50 mL before the addition of 8 μL benzonase. After 30 min incubation at 37° C., the tubes are centrifuged at 10,000 g for 15 min at 4° C.

Cesium Chloride Gradient Purification: To achieve the gradient, 10 mL of cesium chloride at a density of 1.3 grams/mL is deposited in ultracentrifuge tubes. A volume of 5 mL of cesium chloride at a density of 1.5 grams/mL is then placed underneath. The supernatant is gently deposited on top of the cesium chloride and the tubes are ultracentrifuged at 28,000 RPM for 24 hours at 20° C. Two bands are observed: the upper band contains the empty capsids and the lower band corresponds to the full capsids. Both strips are collected avoiding the removal of impurities. The sample is mixed with cesium chloride at a density of 1.379 g/mL in a new ultracentrifuge tube and then ultracentrifuged at 38,000 RPM for 72 hours at 20° C. The solid capsid strip is removed.

Concentration and filtration: The removal of cesium chloride from the viral preparation and the concentration are carried out on Amicon®(Merck) filters. On Amicon®(Merck) filters, the vectors are concentrated by ultrafiltration with a cut-off of 100 kDa. Amicon membranes are first hydrated with 14 mL 20% ethanol, centrifuged 2 min at 3000 g, then equilibrated with 14 mL PBS, centrifuged 2 min at 3000 g, and then with 14 mL 1,379 ClCs. The collected solid capsid strip is placed on the filters and centrifuged 4 min at 3000 g. 15 mL PBS 1×+F68 formulation buffer is added, before further filtration 2 min at 1500 g. The three previous steps are repeated 6 more times before recovering the last concentrate. The samples are then filtered at 0.22 μm.

Titration: The vector is then assayed by quantitative PCR.

### 1.9 Statistics

In all statistical analyses, the differences are considered significant at P<0.05 (*), moderately significant at P<0.01 (**) and highly significant at P<0.001 (***), with P=probability. Bar graphs are shown as means+SEM standard deviations. The graphs are made using the GraphPad software.

Analysis of the distribution of fibrosis over the whole heart: In order to ensure that the fibrosis is homogeneous in the heart (H0 hypothesis), the inventors randomly drew 20 values from the 483 fibrosis ratio values. These values were compared 10 to 10 with a Wilcoxon test (Software R) to obtain a p-value. This operation was repeated 1000 times, resulting in 1000 p-values. Among these values some are below 0.05 showing that in some cases our hypothesis of fibrosis invariance is not valid. Out of the 1000 statistical tests, the inventors counted how many gave a value below 0.05. We repeated the entire process 100 times to obtain an average of the percentage for which our HO hypothesis is false. This average is 4%. This means that our hypothesis is valid 96% of the time, and therefore corresponds to an overall p-value of 0.04, which is statistically acceptable.

### 2. Results

### Morphological Evaluation

The overexpression of NRF2 was tested by injecting an AAV9-Tnnt2-mNRF2 vector in DeltaMex5 mice. The 1-month-old mice were injected intravenously at a dose of 2e¹¹ vg/mouse (equivalent to a dose of 1e¹³ vg/kg for a mouse of approximately 20 g) or by PBS. After 3 months of vector expression, the hearts of the mice were ultrasonographed prior to collection. The overall, histological and functional consequences on the heart were then studied.

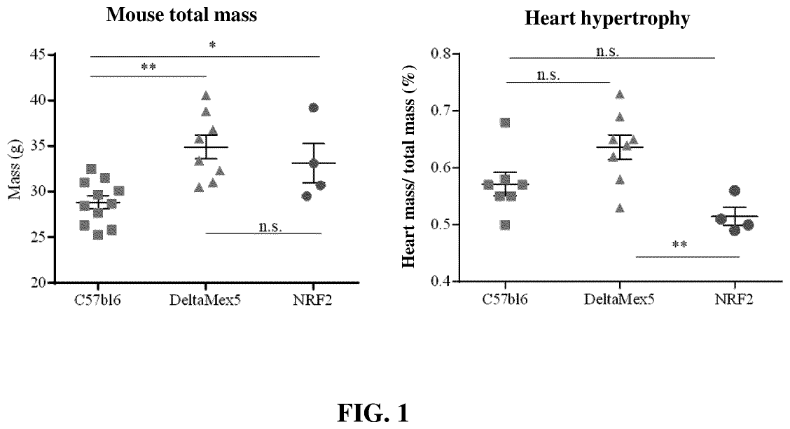

Mouse mass was not significantly decreased in NRF2 vector-treated mice after 3 months (33.13±2.15 g, versus 34.9±1.3 g, n=8) and remained greater than that of C57BL/6 control mice (28.81±0.72, n=11, P=0.03). On the other hand, heart hypertrophy was decreased. Indeed, the ratio of heart mass to total mouse mass was significantly decreased in Nrf2-treated DeltaMex5 mice compared to untreated DeltaMex5 mice (0.51±0.02, n=4 versus 0.63±0.02%, n=8, P=0.005) (FIG. 1).

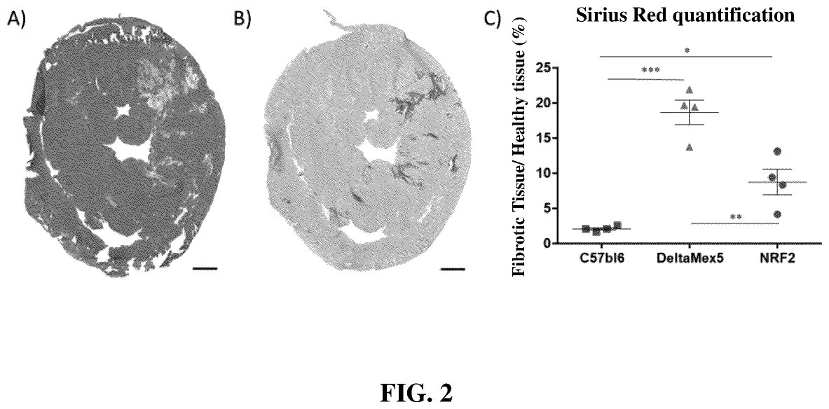

Histological analyses carried out on the hearts of mice show for the HPS stain reveal a clear decrease in the damaged tissue in the treated mice. Sirius Red staining still shows the presence of fibrotic tissue in the hearts of treated mice, but in smaller quantities. Quantification of total tissue fibrosis by Sirius Red collagen staining confirms that the fibrosis rate is decreased in the hearts of treated DeltaMex5 mice compared to untreated DeltaMex5 mice (8.74±1.85% versus 18.68±1.74%, P=0.0078, n=4), although it is still higher than the fibrosis rate in the hearts of C57BL/6 mice (2.08±0.17%, P=0.0115, n=4) (FIG. 2).

The overexpression of the Nrf2 gene in DeltaMex5 model developed by the inventors using an AAV9 viral vector and a cardiac promoter showed a significant improvement in cardiac fibrosis and the enlarged heart.

1-15. (canceled)

16. A method of treating dilated cardiomyopathies in a patient in need thereof, comprising administering to the patient a therapeutically effective amount of a NRF2 activator.

17. The method according to claim 16, wherein the NRF2 activator is a nucleic acid construct comprising a transgene encoding human NRF2 or a variant thereof.

18. The method according to claim 17, wherein said nucleic acid construct comprises a cardiac promoter selected from the group consisting of: human cardiac troponin T promoter (TNNT2), alpha myosin heavy chain promoter (α-MHC), myosin light chain 2v promoter (MLC-2v), myosin light chain 2a promoter (MLC-2a), CARP gene promoter, alpha-cardiac actin promoter, alpha-tropomyosin promoter, cardiac troponin C promoter, cardiac myosin-binding protein C promoter, sarco/endoplasmic reticulum Ca²⁺ ATPase (SERCA) promoter, desmin promoter, MH promoter, CK8 promoter and MHCK7 promoter.

19. The method according to claim 17, wherein said nucleic acid construct comprises a human cardiac troponin T promoter.

20. The method according to claim 17, wherein the nucleic acid construct is packaged into a viral particle.

21. The method according to claim 17, wherein the nucleic acid construct is packaged into an adeno-associated viral (AAV) particle.

22. The method according to claim 17, wherein the nucleic acid construct is packaged into an AAV particle and further comprises 5′-ITR and 3′-ITR of AAV-2 serotype or a 5′ITR and a 3′ITR corresponding to the serotype of the selected AAV particle.

23. The method according to claim 17, wherein the nucleic acid construct is packaged into an AAV particle comprising an AAV capsid protein derived from AAV serotypes selected from the group consisting of: AAV-1, 6, 8, 9 and AAV9.rh74 serotypes.

24. The method according to claim 17, wherein the nucleic acid construct is packaged into an AAV particle comprising an AAV capsid protein derived from AAV-9 serotype or AAV9.rh74.

25. The method according to claim 17, wherein the nucleic acid construct is packaged into a viral particle that is administered intravenously.

26. The method according to claim 16, wherein said dilated cardiomyopathy is a genetically induced cardiomyopathy caused by mutation(s) in a gene selected from the group consisting of :
laminin, emerin, fukutin, fukuti-related protein, desmocollin, plakoglobin, ryanodine receptor 2, sarcoplasmic reticulum ca(2+) ATPase 2 isoform alpha, phospholamban, lamin a/c, dystrophin, telethonin, actinin, desmin, sarcoglycans, titin, myosin RNA-binding motif protein 20, BCL-2 associated athanogene 3, desmoplakin, sodium channels, cardiac actin, cardiac troponin and tafazzin.

27. The method according to claim 16, wherein said dilated cardiomyopathy is a genetically induced dilated cardiomyopathy caused by mutation in titin or dystrophin gene.

28. The method according to claim 16, comprising administering a pharmaceutical composition comprising the NRF2 activator and a pharmaceutical excipient.
